(19) United States
(12) Patent Application Publication
(10) Pub. No.: US 2005/0148853 A1
(43) Pub. Date: Jul. 7, 2005

(54) Method for supporting navigation of a medical instrument, in particular of a catheter

(76) Inventor: Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/013,882

(22) Filed: Dec. 16, 2004

(30) Foreign Application Priority Data
Dec. 17, 2003 (DE) 10359317.9

(51) Int. Cl. A61B 5/05

(52) U.S. Cl. 600/407

(57) ABSTRACT

Method for the selective navigation of a medical instrument, in particular of a catheter, invasively inserted into a hollow organ of the human body or the body of an animal to a pathological site in the hollow organ, in which method on the basis of a prior, first image display recorded by means of a non-invasive examination procedure of at least a part of the hollow organ the position of one or more pathological sites is determined and the image display is reproduced during subsequent navigation of the instrument together with a continuous angiographically recorded angiography image display of at least a part of the hollow organ in which the tip of the instrument is located.

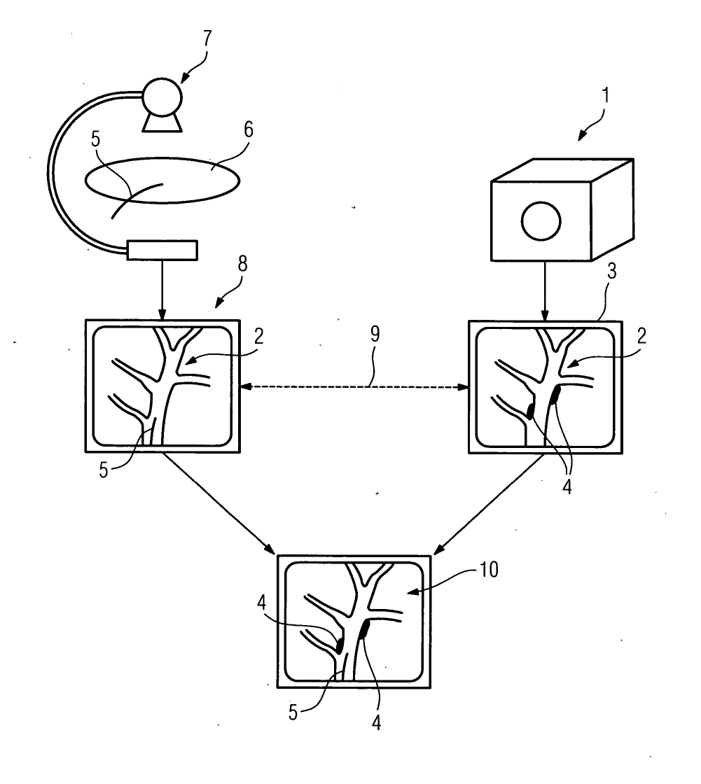

### CROSS REFERENCE TO RELATED APPLICATIONS

This application claims priority to the German application No. 10359317.9, filed Dec. 17, 2003 which is incorporated by reference herein in its entirety.

### FIELD OF INVENTION

The invention relates to a method for the selective navigation of a medical instrument, in particular of a catheter, inserted invasively into a hollow organ of the human body or the body of an animal to a pathological location in the hollow organ.

### BACKGROUND OF INVENTION

Coronary heart disease is the result of underlying coronary arteriosclerosis and manifests itself in symptoms such as stable and unstable angina, heart attacks and sudden cardiac death. It is thus the main cause of death in western industrialized countries and is responsible for very high costs in the healthcare sector.

In approx. 85% of all cases a certain form of arteriosclerosis, so-called “Vulnerable Plaques”, is responsible for acute coronary symptoms. An essential difference between these “Vulnerable Plaques” and stable, calcified stenoses is a very slight, usually non-existent obstruction of blood flow in the coronary arteries. This is due to so-called “positive remodeling”. In the case of this positive remodeling, the plaque does not initially expand into the lumen of the vessel but into the vascular wall itself. As a result, the “Vulnerable Plaques” cannot be detected or diagnosed in conventional angiography, and differentiation of the different plaque pathologies is not possible.

### SUMMARY OF INVENTION

To this end, different invasive imaging methods were developed to take advantage of the different properties of the “Vulnerable Plaques” for diagnosis, as well as to display the morphology in high resolution. Mention is made of invasive imaging examination methods such as, for example, intravascular ultrasound examination (IVUS) or optical coherence tomography (OCT), while non-invasive examination methods include, for example, magnetic resonance and computer tomography examination. The main disadvantage of the interesting, invasive OCT method is that the blood from the vascular parts for examination needs to be removed by flushing and/or using a balloon which prevents the flow of blood. Furthermore, a lengthy examination of all possible vascular branches is necessary as the “Vulnerable Plaques” cannot be located by means of the X-ray angiography monitoring performed in parallel to register catheter movement. This leads to prolonged examination times in the catheter laboratory, when using the invasive imaging method the risk to the patient increases, and furthermore there is increased exposure to radiation. The main disadvantage of non-invasive methods, on the other hand, is among other things the lack of site resolution, so that a statement about the essential criterion for assessment of the risk of an acute occurrence, namely the thickness of the fibrous plaque cover, is in particular not possible. However, precisely from a diagnostic point of view, it is important to be able to assess the actual risk of an occurrence resulting from tearing of the plaque cover.

An object of the invention is therefore to specify a method which permits simple navigation and as a result rapid examination and location of the relevant pathological sites, especially the “Vulnerable Plaques”, to lessen the risk to the patient and reduce exposure to radiation during necessary execution of the invasive method to examine the hollow organ.

This object is achieved by the claims. To solve the problem, it is envisaged that on the basis of an initial image display recorded beforehand by means of a non-invasive examination procedure of at least a part of the hollow organ, the position of one or more pathological sites is determined and the image display is reproduced during subsequent navigation of the instrument together with a continuous angiographically recorded angiography image display of at least a part of the hollow organ in which the tip of the instrument is located.

The solution according to the invention is for successive use of a non-invasive examination procedure such as, for example, a magnetic resonance or computer tomography examination and an invasive examination procedure (such as, for example, IVUS or O CT examinations) which each provide separate image displays, containing different statements, of the hollow organ or of the recorded part. Via the first, non-invasively recorded image display, e.g. the MR or the CT image, the “Vulnerable Plaques” can be located in the image by means of larger inflammatory processes, i.e. information is obtained about the position of the pathological sites in the hollow organ. This image display is reproduced during the invasive examination. This means that the doctor providing the treatment has continuous knowledge of the precise location of the sites of interest to which the catheter is to be routed. Navigation is now made still easier for him by the performance of an X-ray angiography control and the angiography image also being displayed continuously in parallel with the movement of the catheter. In this way the doctor can without further action immediately compare the actual position of the catheter tip and the actual location of a “Vulnerable Plaque” toward which to move. The movement of the instrument can be controlled manually, computer-aided or entirely computer-controlled.

Owing to the possibility of considerably more efficient and more specific navigation, the risk always associated with the invasive examination method is considerably reduced for the patient as it is no longer necessary to examine all the possible vascular branches in order to locate the plaque, since a pathological site can be approached specifically. This means that the duration of the invasive examination is considerably reduced. Inevitably this also applies to the duration of exposure to radiation resulting from the angiography control examination. In order to make recording of the pathological sites easier for the doctor to locate on the monitor in the first image display, it is expedient if the pathological site or sites are highlighted in the first image display. This highlighting, e.g. by means of color highlighting, can for example be done by the user himself by first assessing the image recorded non-invasively before the invasive examination and highlighting the pathologically relevant sites on the monitor, for example, using the usual monitor cursor and suitable editing software. As an alternative to manual highlighting, it is also conceivable to locate the pathological sites automatically in the image using a suitable image analysis system based on suitable analysis algorithms, i.e. highlighting is done automatically, insofar as the image display or pathology permits this.

With regard to simple recording of the image information in the first image display as well as the angiography image display, an advantageous development of the invention envisages that the multidimensional form of the first image display corresponds to that of the angiography image display. The angiography images are two-dimensional, for which reason the first display, obtained from, for example, the MR or the CT examination, is also two-dimensional. If the MR or CT data record is three-dimensional, the corresponding two-dimensional projection is calculated and displayed from it as appropriate. Furthermore, a position data record using a position recording system can be made, for example, for each individual angiography image, for which purpose a corresponding position sensor is used in the catheter, which permits the recording of a movement by preferably six degrees of freedom (in x, y and z direction of the coordinate axes as well as by the respective rotation around this axis). From this the spatial location of the catheter in the MR or CT display is possible, which can then also be indicated as a 3D-image in addition.

A particularly advantageous development of the invention envisages registering the first image display and the angiography image display with one another and displaying them merged together. After this the angiography image is overlaid on the MR or CT image or on the respective cutting plane display. In this way, the doctor obtains information about the anatomy of the examination area from the MR or CT display, and he obtains the information about the actual position of the catheter tip from the overlaid angiography image display. On account of the registration of both image displays, it is always possible to insert the angiography image precisely into the MR or CT image, even where there is constant catheter movement and as a result a constant change in the recording position, and thus enable a constantly adapted, “co-moved” fusion display.

Registration can take place according to a first inventive alternative on the basis of anatomical landmarks which are available in both image displays. The image data records which must be registered with one another are examined for corresponding anatomical structures such as, for example, special vascular branches, etc. via suitable image analysis software or suitable analysis algorithms and registered with one another on the basis of these conformities. This means that, on the basis of these anatomical landmarks, it is possible to determine the mapping rule bringing about registration which makes it possible to map the pixels of both the two-dimensional images or the voxels of the three-dimensional displays to one another respectively.

As an alternative to registration on the basis of the anatomical landmarks, registration can take place via position data continuously obtained for each recorded angiography image display, which indicates the position and/or orientation of the instrument in the coordinates system of the position recording system, which provides the basis for the registration of the position data with the position data recorded for the first image display in the coordinates system of the examination procedure. As already described, there is the possibility of recording the spatial position data for each angiography image display. Such data already forms the basis for the MR or CT image record, i.e. there too the respective position data record for each individual image is known. In this way it is possible to register both the coordinates systems with one another and therefore to obtain the mapping rule.

### BRIEF DESCRIPTION OF THE DRAWING

Further advantages, characteristics and details of the invention are revealed in the described embodiment which follows as well as on the basis of the drawing.

In this, the basic sequence of the inventive method is shown in the form of a schematic representation.

### DETAILED DESCRIPTION OF INVENTION

Via a first examination procedure 1, here a magnetic resonance system, a first image data record of the examination area, in the example shown of a vascular tree 2, is initially recorded. The image, whether a two-dimensional or three-dimensional image, is output on a monitor 3 in order to highlight pathologically relevant sites 4 in the vascular tree 2. These sites may, for example, be the positions of “Vulnerable Plaques”. For highlighting, for example, a monitor cursor can be used, via which the corresponding positions can be examined, which can then, for example, be highlighted in color. Alternatively, it is also conceivable that this highlighting and the prior localization of these sites take places automatically via the image processing equipment.

This data record is recorded before the invasive examination, and is then made available in the catheter laboratory during the invasive examination.

Within the framework of the invasive examination, a catheter 5 is introduced into the vascular tree 2 of the patient 6, who is only shown symbolically here. During movement of the catheter, X-ray angiography takes place continuously using X-ray angiography equipment 7, i.e. individual images are recorded continuously which show the catheter tip in the vascular tree 2. This is displayed in the form of the monitor 8, on which the currently recorded vascular tree 2 with the catheter 5 located therein is displayed as an example.

As described, the 2D projection image calculated from the 3D MR record and the recorded 2D angiography image both show the same hollow organ, namely the vascular tree 2. On the one hand, there is now the possibility of showing the doctor providing the treatment both displays on one monitor or on two monitors in parallel. However, as shown in the FIGURE, a merger of the two image displays takes place, to which end these are registered with one another beforehand, as shown by the double arrow 9. Within the framework of this registration a mapping rule is obtained which makes it possible to directly assign the pixels or voxels of the respective image displays to one another and to map them to each other. Registration can take place on the basis of anatomical landmarks by means of suitable image analysis, to which end the respective images are examined for corresponding, conspicuous anatomical structures. Alternatively, there is the possibility of undertaking registration on the basis of the recorded position data for each individual image of the X-ray angiography in a coordinates system of a position recording system not shown in greater detail, which is registered with position data of the MR coordinates system available in the MR data record. Both registration methods are sufficiently known and need not be described in greater detail.

In any case, registration offers the possibility of merging both individual image displays with each other and providing a merged image 10. In this, on the one hand, the pathological sites 4 are visible, and this image information derives from the MR image display. These sites are not visible in the angiography image display. However, the image information with regard to the catheter 5 comes from the angiography display. As the merged image shows, the precise location of the catheter 5 with regard to the position of the pathological sites 4 can be obtained in the merged image. Simple navigation of the catheter 5 in the vascular tree 2 is thus possible to any pathologically relevant and highlighted site. This can then be approached easily and quickly and if necessary, also be treated in situ.

1-5. (canceled)

6. A method for supporting navigation of a medical instrument inserted into a hollow organ of a human or animal body to a pathological site in the hollow organ, comprising:
recording a first image of at least a part of the hollow organ by a medical non-invasive image recording device;
locating the pathological site using the first image;
displaying a merged image including the first image and a second image continuously recorded angiograhpically during navigation, the second image including at least a further part of the hollow organ in which a tip of the medical instrument is currently present, the tip visualized in the second image; and
indicating the pathological site in the first image.

7. The method according to claim 6, wherein locating and indicating the pathological site in the first image is carried out manually by a user.

8. The method according to claim 6, wherein locating and indicating the pathological site in the first image is carried out automatically by an image analysis system.

9. The method according to claim 6, wherein indicating the pathological site in the first image includes marking and highlighting the pathological site.

10. The method according to claim 6, wherein the medical instrument is a catheter.

11. The method according to claim 6, wherein a the first image and the second image have the same dimension.

12. The method according to claim 11, wherein the first image is generated by transforming a three-dimensional image data set into a two-dimensional data set.

13. The method according to claim 11, wherein the first image and the merged image are three-dimensional and the second image is positioned within the first image according to a current position of the tip acquired by a sensor system relative to a three-dimensional coordinate system.

14. The method according to claim 6, wherein the first image and the second image are registered with each another and displayed in form of the merged image.

15. The method according to claim 14, wherein the registration is based on anatomical landmarks present in the first and second images.

16. The method according to claim 14, wherein, during recording of the second image, a position or orientation of the medical instrument is recorded relative to a coordinate system of a position recording system and the registration includes linking the recorded position or orientation respectively with further position data captured relative to a coordinate system assigned to the medical non-invasive image recording device.
